(19) United States
(12) Patent Application Publication
(10) Pub. No.: US 2017/0172570 A1
(43) Pub. Date: Jun. 22, 2017

(54) MULTIPLE PANEL SUTURE DISPENSING CARD AND METHOD FOR USING THE SAME

(71) Applicant: SURGICAL SPECIALTIES CORPORATION, Braintreet, MA (US)

(72) Inventors: Angela Wentling, Perkiomenville, PA (US); David Mucko, Lancaster, PA (US); Mark Hoyt, Birdsboro, PA (US)

(73) Assignee: SURGICAL SPECIALTIES CORPORATION, Braintree, MA (US)

(21) Appl. No.: 15/125,953

(22) PCT Filed: Mar. 13, 2015
(86) PCT No.: PCT/US2015/020548

§ 371 Date: Sep. 13, 2016

Related U.S. Application Data
(60) Provisional application No. 61/952,660, filed on Mar. 13, 2014.

(51) Int. Cl. A61B 17/06; A61B 50/20; B65D 5/50; B65B 63/04; B65D 5/02; B65D 5/42; A61B 17/04; B65B 5/02

(52) U.S. Cl. CPC A61B 17/06138; A61B 17/0483; A61B 50/20; B65B 5/02; B65B 63/04; B65D 5/02; B65D 5/4266; B65D 5/50; A61B 2017/06142

(57) ABSTRACT

The present disclosure is directed towards an apparatus comprising a card adapted to mount on a fixture and further adapted to fold into a box-shaped package with at least one wall and an internal cavity adapted to contain a suture, the card including a base and a plurality of tabs extending from the base, wherein one of the plurality of tabs includes a needle nest and one of the plurality of tabs is adapted to unfold and open one wall for removing the suture.

### CROSS-REFERENCE TO RELATED APPLICATIONS

This application claims the benefit under 35 U.S.C. §119(e) of U.S. Provisional Patent Application No. 61/952,660, filed Mar. 13, 2014, which application is hereby incorporated by reference in its entirety.

### BACKGROUND

Technical Field

This disclosure relates to suture packaging devises and methods for packing sutures. More particularly, this disclosure relates to foldable suture packages and methods for packaging sutures in foldable suture packages.

Description of the Related Art

Wound closure devices such as sutures, staples, and tacks have been widely used in surgical procedures in humans and animals for closing wounds, repairing traumatic injuries or defects, joining tissues together, attaching foreign elements to tissues, repositioning tissues to new anatomical locations and a myriad of other purposes.

Sutures are also often used as wound closure devices. Sutures typically consist of a filamentous suture thread attached to one or more needles with a sharp point. Suture threads can be made from a wide variety of materials including bioabsorbable (i.e., that break down completely in the body over time), or non-absorbable (permanent; non-degradable) materials. Absorbable sutures have been found to be particularly useful in situations where suture removal might jeopardize the repair, or where the natural healing process renders the support provided by the suture material unnecessary after wound healing has been completed; as in, for example, completing an uncomplicated skin closure. Non-degradable (non-absorbable) sutures are used in wounds where healing may be expected to be protracted or where the suture material is needed to provide physical support to the wound for long periods of time; as in, for example, deep tissue repairs, high tension wounds, many orthopedic repairs and some types of surgical anastomosis.

Like any long flexible filament, sutures may tangle during removal from their packaging, when preparing them for use, or during use. Because of the tendency for long flexible filaments to tangle, manufacturers and medical practitioners use great caution to prevent tangling during the packaging process and use of sutures. The handling of sutures and their attached needles also complicates the packaging and use of sutures.

Improperly packaged sutures may place the needles in a position where it is more likely to cause harm to medical practitioners, and some packaging device designs and methods for packaging may place the needles in less secure or less safe positions.

What is needed is a packaging device that safely and securely holds the suture and its needles and provides for its easy and safe removal. In addition, safe, easy, and efficient methods for loading and removing sutures from their packaging are also needed.

### BRIEF SUMMARY

The present disclosure is directed towards an apparatus for holding a suture. The apparatus comprises a card including a base and a plurality of panels projecting outwardly from the base, the panels being adapted to fold to form the card into a box-shaped package. The base is adapted to mount on a fixture when the card is in an unfolded configuration for receiving the suture. The box-shaped package includes a plurality of walls enclosing an internal cavity for containing the suture. One of the plurality of panels includes a slit adapted to retain a portion of the suture proximate a needle while the suture is being placed in the apparatus. One of the plurality of panels includes a needle nest adapted to capture the needle after the remainder of the suture has been placed into the apparatus, and one of the plurality of panels is adapted to facilitate bending whereby a user can open a portion of the box-shaped package to access the needle and remove the needle and suture therefrom.

The present disclosure is also directed towards a method comprising the steps of: mounting a suture packaging card onto a mounting fixture with a portion of the mounting fixture passing through the suture packaging card, wrapping a suture into a coil around the portion of the mounting fixture that was passed through the suture packaging card, securing the suture coil to the suture packaging card, removing the suture packaging card from the mounting fixture, securing a suture needle to the suture packaging card, forming the suture packaging card into a box shape, and securing the suture packaging box

The present disclosure is also directed towards a system for packaging a suture. The system comprising a mounting fixture comprising a plurality of pins and a suture packaging card having a base comprising a plurality of apertures. The pins on the mounting fixture being configured to pass through the apertures in the base of the suture packaging card and to receive a coil of the suture material to be packaged within the suture packaging card. The suture packaging card comprising a plurality of panels projecting outwardly from the base. The panels being adapted to fold over the base to form the card into a box-shaped package for retaining the suture.

### BRIEF DESCRIPTION OF THE SEVERAL VIEWS OF THE DRAWINGS

FIG. 1 shows a top view of an embodiment of a folding suture package;

FIGS. 2A and 2B show isometric and cross-sectional views, respectively, of a suture wrapping and packaging fixture;

FIG. 3 shows one step of one method for wrapping and packing a suture in a folding suture package;

FIG. 4 shows another step of one method for wrapping and packing a suture in a folding suture package;

FIG. 5 shows another step of one method for wrapping and packing a suture in a folding suture package;

FIG. 6 shows another step of one method for wrapping and packing a suture in a folding suture package;

FIG. 7 shows another step of one method for wrapping and packing a suture in a folding suture package;

FIG. 8 shows another step of one method for wrapping and packing a suture in a folding suture package;

FIG. 9 shows one step of one method for opening and removing a suture from a folding suture package; and

FIG. 10 shows another step of one method for opening and removing a suture from a folding suture package.

### DETAILED DESCRIPTION

In the following description, certain specific details are set forth in order to provide a thorough understanding of various embodiments of the invention. However, one skilled in the art will understand that the invention may be practiced without these details. In other instances, well-known structures and steps associated with suture packaging have not been shown or described in detail to avoid unnecessarily obscuring descriptions of the embodiment of the invention.

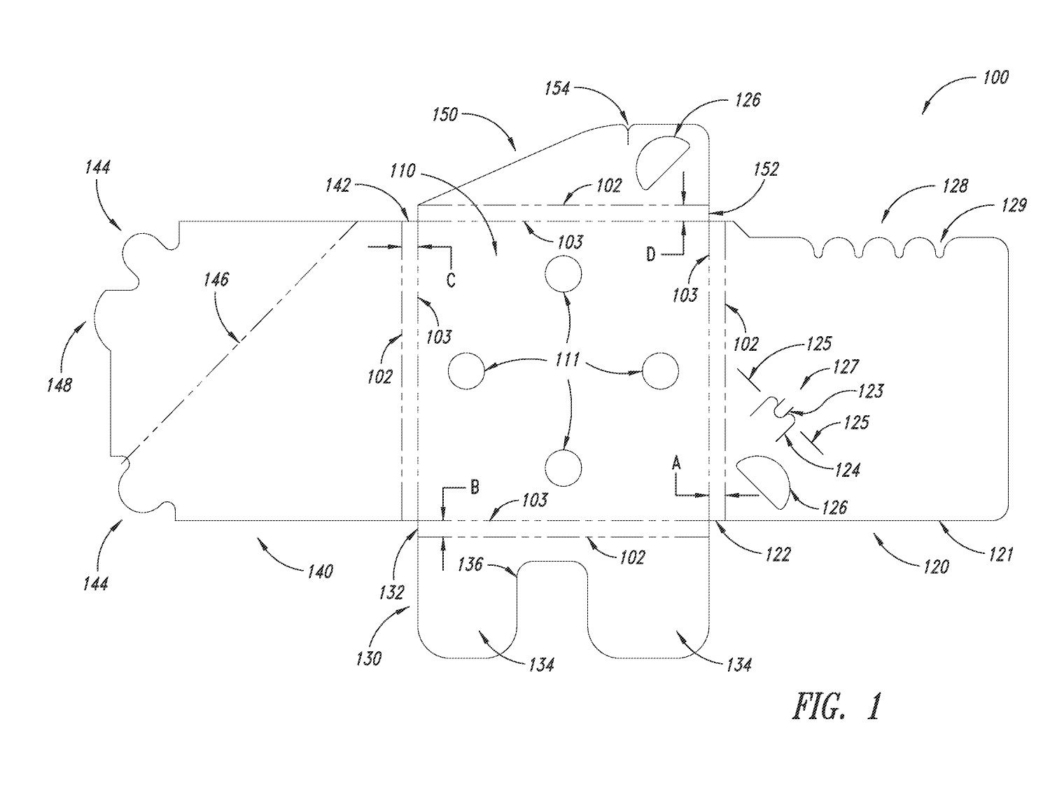

FIG. 1 shows an embodiment of a foldable package for packing and transporting a suture. The package may be comprised of a card 100. Throughout this disclosure the terms card and package generally refer to a foldable package for packing and transporting a suture, with the term “card” generally referring to the foldable package for packing and transporting a suture in an unfolded or unpacked state and the term “package” generally referring to a card in at least a partially closed or packed state.

The card 100 may be made from paper or other suitable materials. In some embodiments the card 100 is made from 9-point white surgical kraft paper.

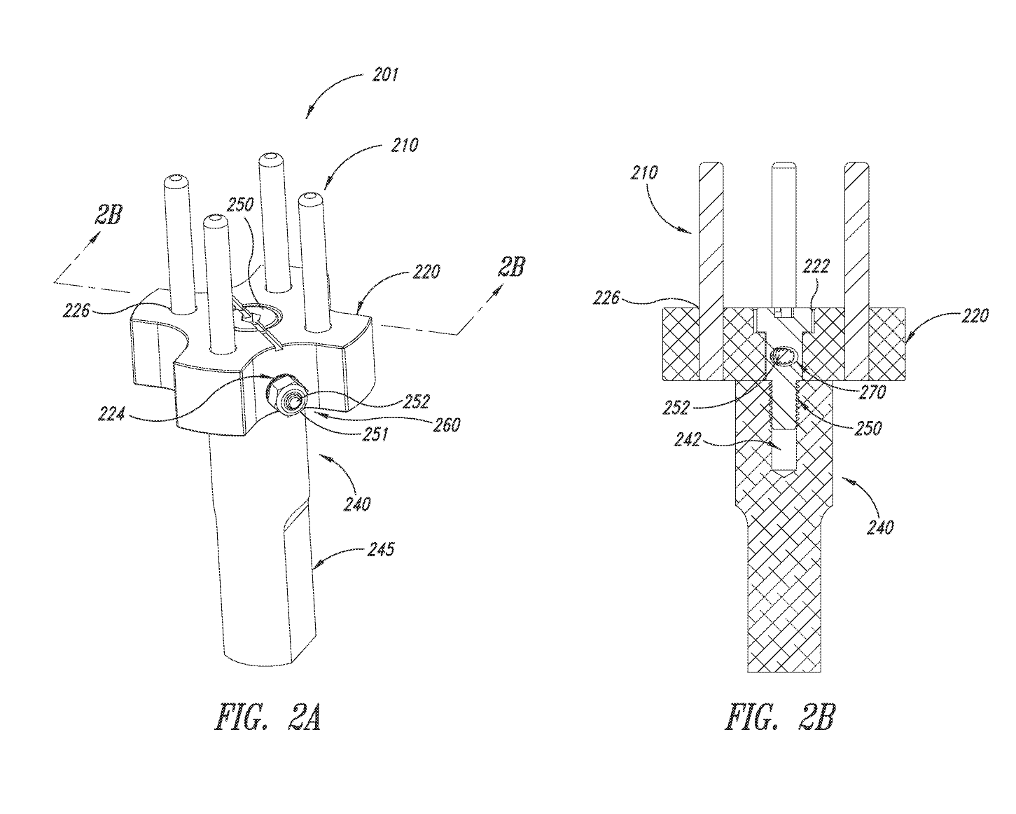

The card 100 may include a base 110, which may include one or more fixture holes 111. The fixture holes 111 may be apertures cut, punched, or otherwise formed through the base 110. The fixture holes 111 allow one or more fixture pins, such as fixture pins 210, shown in FIG. 2, to pass through the holes 111.

Although the embodiment shown in FIG. 1 depicts a card 100 with four round fixture holes 111, in some embodiments the fixture holes 111 may be other shapes, in other configurations, and/or in other numbers. For example, in some embodiments three holes may be used. In some embodiments, for example, in an embodiment with one fixture hole 111, the fixture hole may have a ‘+’ and ‘X’ shape.

In an unfolded position, as shown in FIG. 1, the card 100 has four panels 120, 130, 140, and 150 extending from a base 110. Each panel 120, 130, 140, and 150 may include fold indicators 102 and 103 and a wall 122, 132, 142, and 152. The distance between the fold indicators 102 and 103 determine the height of the corresponding wall 122, 132, 142, and 152. For example, fold indicators 102 and 103 of panel 120 delineate the upper and lower edge of wall 122.

In some embodiments the fold indicators 102 and 103 are score lines. In some embodiments the score lines are formed by using an embossing tool, such as an embossing die, which applies a force along the fold indicator 102 and 103 to deform or create an indentation in the card 100 along the fold indicator 102 and 103. The deformation or indentation weakens the card 100 along the fold indicator 102 and 103 and facilitates folding a panel along the fold indicator 102 and 103.

In some embodiments, the fold indicators 102 and 103 are formed by making a shallow cut or line of perforations in the card 100. A shallow cut does not penetrate through the entire card 100, but penetrates less than all the way through the card 100 to weaken it along the fold indicators 102 and 103. A line of perforations, on the other hand, is a series of small holes or cuts through the card 100 along the fold indicators 102 and 103 to weaken it along the fold indicators 102 and 103. In some embodiments the fold indicators 102 and 103 may be a combination of deformations, indentations, cuts, and/or lines of perforations.

The walls 122, 132, 142, and 152 may have similar or different heights when formed. For example, wall 122 may have a height ‘A,’ while wall 132 may have a height ‘B.’ The difference in height of the walls 122, 132, 142, and 152 may account for potential differences caused by manufacturing tolerances, for the thickness of the packaged sutures, or for the additional thickness of each panel 120, 130, 140, and 150.

As discussed below, each panel is folded on top of each successive panel as the card 100 is formed into a box shape with an internal cavity, a top, a bottom, and a wall or walls extending between the top and bottom. To create a well-formed box, each wall should have a height that accounts for the material it covers such that it lays flat over the material. For example, in some embodiments, after positioning the sutures on the base 110, an operator may fold a first panel 130 over the sutures. In such an embodiment the height ‘B’ of wall 132 should account for the thickness of the sutures.

The operator may then fold a second panel 120 over the top of the first panel 130 and the sutures. In this case, the height ‘C’ of wall 122 should account for the thickness of the sutures and of the first panel 130. The height of the other walls would likewise account for the thickness of material over which a corresponding panel is folded.

In some embodiments, all wall heights are the same. In addition, in some embodiments the card 100 may not include any walls. In such embodiments, the card 100 may only include one fold indicator 102 or 103 for each panel 120, 130, 140, and 150 and the panels 120, 130, 140, and 150 may deform or bend to cover the packaged material or the other panels.

Each panel 120, 130, 140, and 150 may include one or more additional fold indicators. For example, the left panel 140 includes fold indicator 146. As discussed in more detail below, this fold indicator 146 may facilitate opening the package. For example, by creating a weak point in the panel 140, the fold indicator 146 may make it easier to bend or fold the panel and facilitate closing or opening the panel 140.

Each panel may include one or more lift tabs 148 to facilitate lifting a panel, such as panel 140 and opening the package 100.

Each panel may also include one or more cutouts. For example, the illustrated bottom panel 130 includes a cutout 136 to facilitate folding the panel 130 over a suture while the card 100 is loaded on a fixture, for example fixture 201 of FIG. 2. In some embodiments, the cutout 136 is of a shape and size that an operator may fold the panel 130 into a closed or packaged position while still on the fixture without the panel interfering with a fixture pin, for example fixture pin 210 of FIG. 2.

In some embodiments, the panels may have more than one cutout. In some embodiments, rather than removing a portion of a panel to create a cutout, the cutout may be a slit or slot through the panel that allows the panel to deform over a fixture pin or other obstruction when put in a closed or packed position.

Each panel may also include one or more suture holding tabs 134. The suture holding tabs 134 may hold the packaged suture in place during the packing operation.

Each panel may also include one or more locking slots. For example, the right panel 120 includes a locking slot 126. The illustrated locking slots 126 are D-shaped or in the shape of a semi-circle or half-moons as shown in FIG. 1. The shape can facilitate the easy insertion and removal of locking tabs, for example, the locking tabs 144. The locking tabs 144 also hold the card 100 securely closed.

In some embodiments, the locking slots 126 may have a different shape. For example, the locking slots could have a rectangular or triangular shape. In some embodiments, the locking slots 126 could even be a slit.

Each panel and the base may also include one or more needle nests. For example, panel 120 includes a needle nest 127. The needle nest 127 is comprised of opposing and interleaved fingers 123 and 124 and one or more slits 125.

The needle nest 127 is adapted to hold one or more suture needles between the set of opposing fingers 123 and 124. The opposing fingers 123 and 124 are opened by bending the panel 120 along a line extending through the middle of the needle nest 127 and the slits 125. The slits 125 are similar to the fold indicators 102, 103, and 146 in that they facilitate the folding or bending of a panel. In particular, slits 125 facilitate the substantially elastic bending of the right panel 120 along a line running between the opposing fingers 123 and 124. This bending causes the fingers 123 and 124 to separate. The separation of the opposing fingers 123 and 124 creates a cavity into which an operator may place one or more suture needles.

After the operator places the needle or needles in the cavity they may remove the bending force. This may cause the opposing fingers 123 and 124 to close around the suture needle or needles, securing it in place.

Each panel may also include one or more suture positioning structures. For example, panel 120 includes suture positioning structure 128. A suture positioning structure 128 is a means for holding or positioning a suture while an operator is not using it. This allows the operator to place an end of the suture proximate to the needle into the positioning structure 128, which holds the suture while the operator parks the needle or needles in the needle nest 127. For example, in some embodiments, the card 100 holds more than one suture. In such embodiments, the operator may place one of the sutures in the positioning structure 128 and a corresponding needle in the needle nest and then proceed to load one or more additional sutures into the card.

The suture positioning structure may be comprised of one or more gates 129. For example, suture positioning structure 128 is comprised of four gates 129. Each gate 129 facilitates holding one or more sutures, but preferably only one suture.

Although panel 120 only shows a suture positioning structure 128 and its associated gates 129 along one edge of the panel 120, in other embodiments the panel 120 may include an additional corresponding or opposing suture positioning structure and associated gates along another edge, for example edge 121.

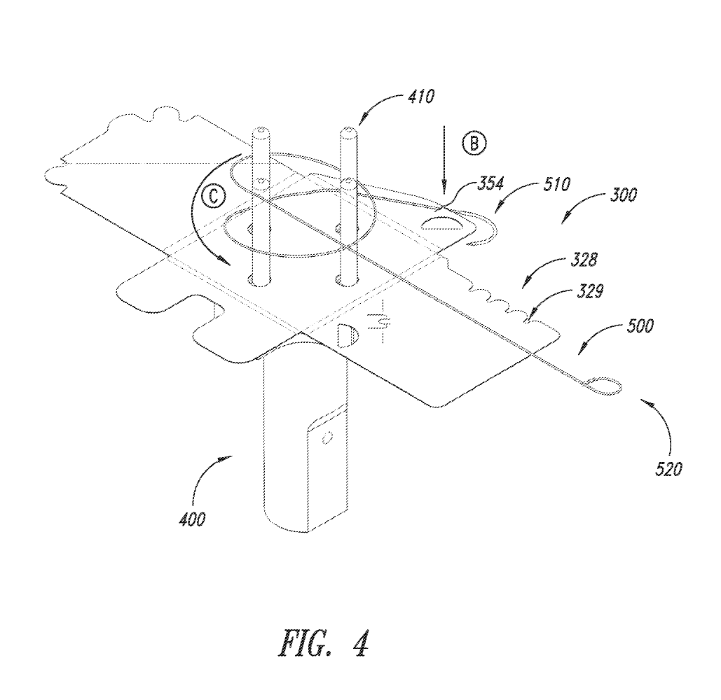

The card 100 may also include one or more wrapping slits. For example, panel 150 includes a wrapping slit 154. The wrapping slit is typically a cut or slot located along an edge of a panel. The wrapping slit 154 provides a place where an operator may secure the needle end of a suture during the wrapping and folding steps of a packaging procedure (see, for example, FIGS. 4, 5, and 6).

When the operator places the needle end of a suture in the wrapping slit 154, the sides of the slit may deform and apply a holding force to the suture. This holding force acts against the suture and helps prevent the needle end of the suture from moving while the operator wraps the suture around the fixture pins 210 and when folding the panels 120, 130, 140, and 150 to close the card 100 around the suture.

The card 100 may also include one or more locking tabs, preferably at least two locking tabs. For example, panel 140 includes two locking tabs 144. As discussed above, each locking tab 144 engages with a corresponding locking slot 126 to releasably hold the panel 140 closed and substantially prevent the closed package 100 from opening.

FIGS. 2A and 2B depict an embodiment of a wrapping fixture. Wrapping fixture 201 may be comprised of a mounting post 240, a mounting plate 220, pins 210, a first coupler 250, and a second coupler 260.

The mounting post 240 includes indexes or flats 245 for coupling the wrapping fixture 201 to a fixture holder, not shown. The flats may interface with the fixture holder to aid in preventing the wrapping fixture from rotating while coupled to or held by the fixture holder.

The first and second couplers 250 and 260 couple the mounting plate 220 to the mounting post 240. The first coupler 260 may pass through an aperture 222 in mounting plate 220 and into a hole 242 in mounting post 240. The vertical hole 242 may be a blind and threaded hole as shown in FIG. 2. In other embodiments the hole 242 may not be blind (e.g. a through hole) and it may not be threaded. When hole 242 is threaded, coupler 250 may be a screw. For example, the coupler may be a shoulder screw as depicted in the embodiment of FIG. 2B.

The second coupler 260 may provide an additional means to couple the mounting plate 220 to the mounting post 240. For example, mounting plate 220 and coupler 250 may include horizontal holes 224 and 270, respectively, through which the coupler 260, such as a machine screw, may pass. In such a configuration, the coupler 260 may prevent the mounting plate 220 from rotating relative to the coupler 250.

In some embodiments, for example, in the embodiment shown in FIGS. 2A and 2B, the coupler 260 may be comprised of a machine screw 252 and a nut 251.

The mounting plate 220 may also include apertures 226 configured to receive pins 210. Pins 210 are configured and/or adapted to receive a suture packaging card, such as card 100 of FIG. 1. In some embodiments, such as the embodiment in FIGS. 2A and 2B, the wrapping fixture 201 includes four pins 210 in a substantially square arrangement such that the four fixture holes 111 of card 100 may slip over the pins 210.

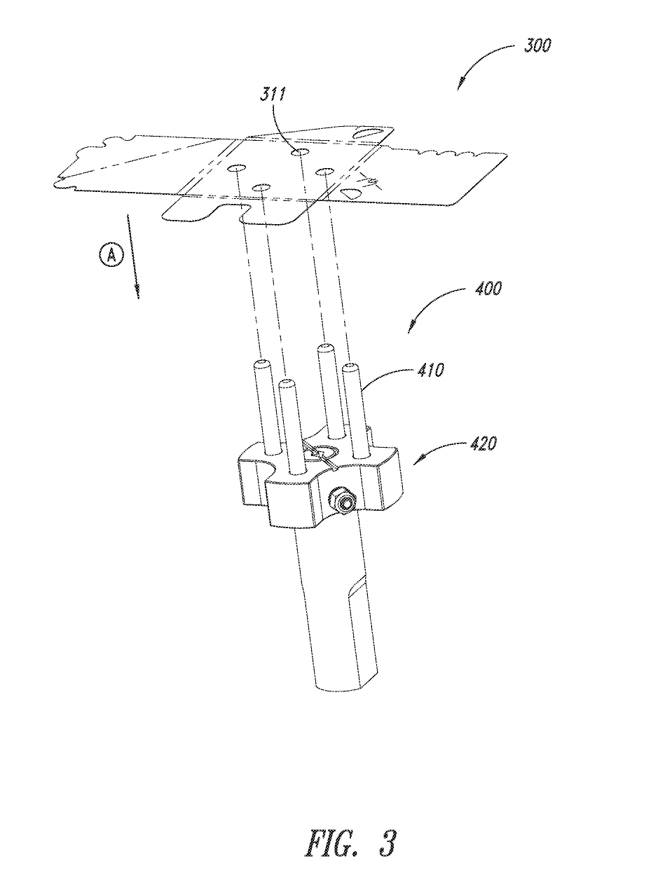

FIGS. 3-9 show exemplary steps and methods for using a wrapping fixture and a card for packaging and using one or more sutures. In this disclosure references to an operator performing an act or step may include the operator performing the act or step manually, such as by manipulating something by hand, or it may also include the automatic or semiautomatic function of a machine performing the same or similar act of step.

FIG. 3 depicts a step of assembling or mounting a card 300 onto a wrapping fixture 400. In an exemplary embodiment, the card 300 is mounted on the wrapping fixture by placing the card's fixture holes 311 over the respective pins 410 and sliding the card 300 down onto an upper surface of the mounting plate 420 in direction A.

In FIG. 4 the needle end 510 of the suture 500 may be secured in a wrapping slit 354 by pushing the suture 500 between the sides of the slit 354 as indicated by arrow B. With the needle end 510 of the suture 500 secure, the loop end 520 of the suture 500 is wrapped around the pins 410 as indicated by arrow C. In some embodiments, an operator manually wraps the suture around a fixed wrapping fixture 400. In other embodiments, the wrapping fixture 400 may rotate while the operator guides the loop end 520 of the suture 500 around rotating pins.

After the first suture 500 is wrapped, additional sutures (not shown) may be wrapped around the pins 410 while the additional needle ends 510 are secured in additional wrapping slits (not shown). In some embodiments, multiple needle ends 510 may be secured in a single wrapping slit 354.

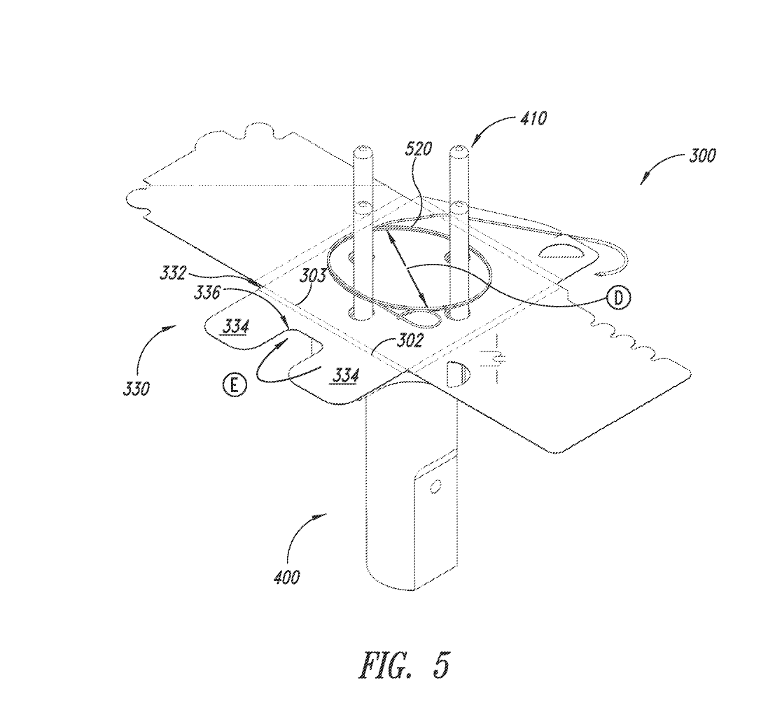

In FIG. 5 the operator organizes the suture coil 520 (which may be comprised of a number of successive loops around the wrapping posts) around the wrapping posts or pins 410 as indicated by the arrows D. In some embodiments, the organizing of the suture coil 520 includes organizing the loops of the suture coil 520 such that each successive loop does not wrap over a previously wrapped loop. In some embodiments, an operator organizes the loops of the suture coil 520 in part by lifting the card 300 at least partially up the pins 410.

The operator may also fold the bottom panel 330 up and over the top of the suture coil 520 as indicated by arrow E. During this step, the bottom panel 330 is bent along fold indicators 303 and 302, thus creating a first wall 332. The operator may then press the suture holding tabs 334 over the top of the suture coils 520. The cutout 336 may prevent the panel 330 and pins 410 from interfering with each other.

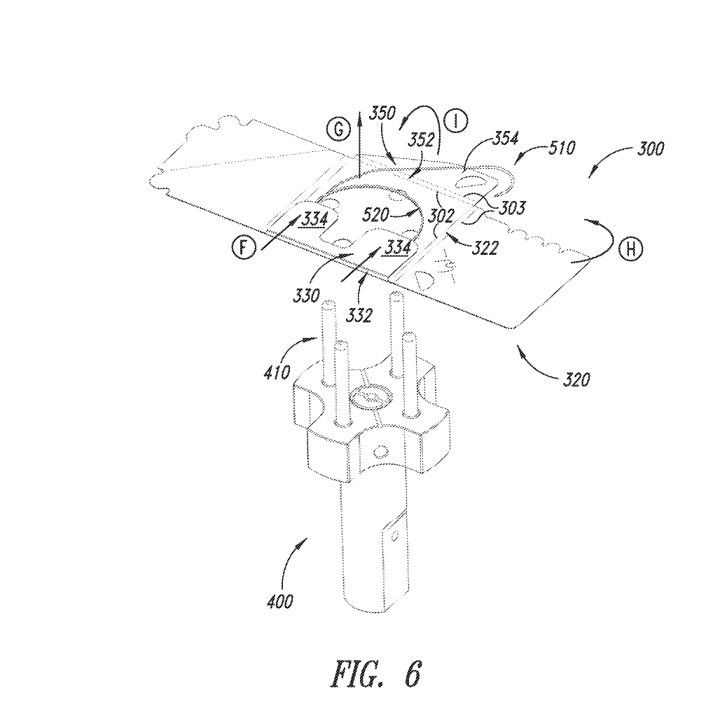

FIG. 6 shows several additional steps of an exemplary embodiment. First, the operator may pinch the suture coil 520 by applying pressure on the suture holding tabs 334 (as indicated by arrows F) and a corresponding location on the underside of the card 300.

With the suture coils 520 held in place, the operator may remove the card 300 from the wrapping fixture 400 by sliding the card 300 up along the pins 410, as indicated by the arrow G.

Next, the operator may fold the right panel 320 up and over the top of the suture coil 520 and the bottom panel 330, as indicated by arrow H. During this step, the panel 320 is bent along fold indicators 303 and 302, thus creating a second wall 322.

Next, the operator may fold the top panel 350 up and over the top of the suture coil 520, the bottom panel 330, and the right panel 320, as indicated by arrow I. During this step, the panel 350 is bent along fold indicators 303 and 302, thus creating a third wall 352. During this process, the needle end 510 is removed from the wrapping slit 354 and placed over one of the positioning gates 329.

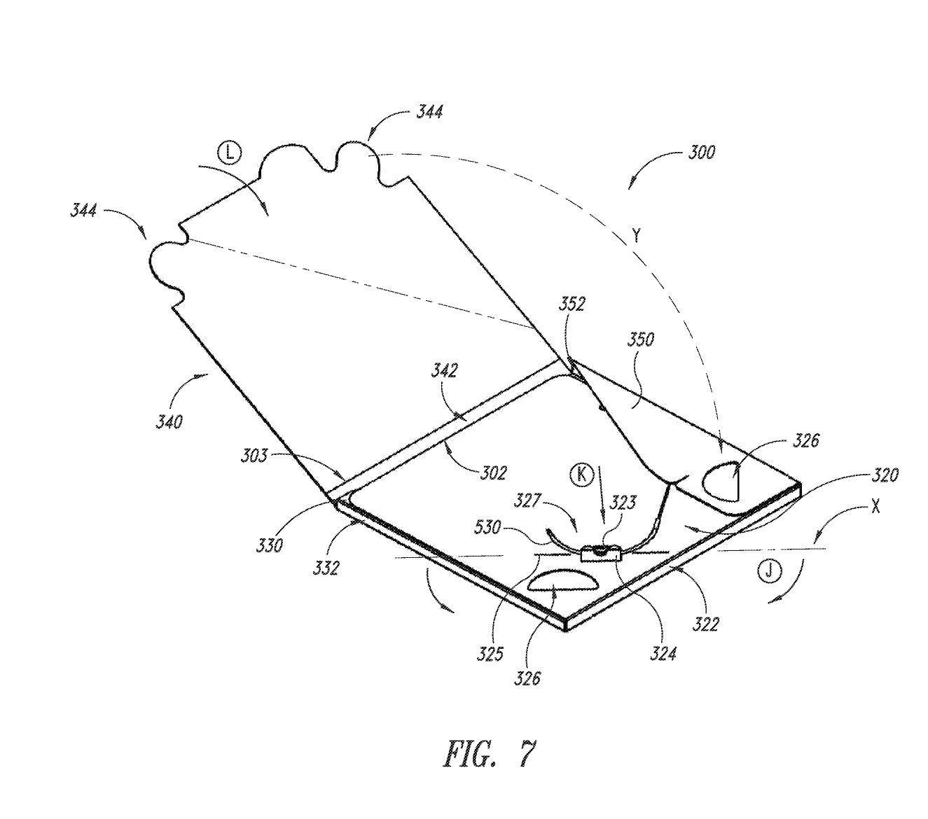

FIG. 7 shows an exemplary embodiment of the card 300 after the operator has folded bottom, right, and top panels 330, 320, and 350, respectively, up over the suture. FIG. 7 also shows the walls 322, 332, and 352, created by folding the panels 320, 330, and 350, respectively.

With the suture needle or needles 530 exposed and laying over the top of the right panel 320, the operator may bend the corner of the card 300 down and along line X, as indicated by arrows J, to separate the fingers 323 and 324 and open the needle nest 327. Aligning the slits 325 with the center of the needle nest 327, along axis X, facilitates folding the corner of the card 300 and opening the needle nest 327. With the needle nest 327 open, the operator may place the needle or needles 530 into the needle nest 327 with the needle facing towards the center of panel 320 and then release the bend on the card 300.

Finally, the operator may fold the left panel 340 over the top of the sutures 500 and the other folded panels 320, 330, and 350 to close the package. The panel 340 is folded along fold indicators 302 and 303, as indicated by arrows L and Y. During this step, a fourth wall 342 is created.

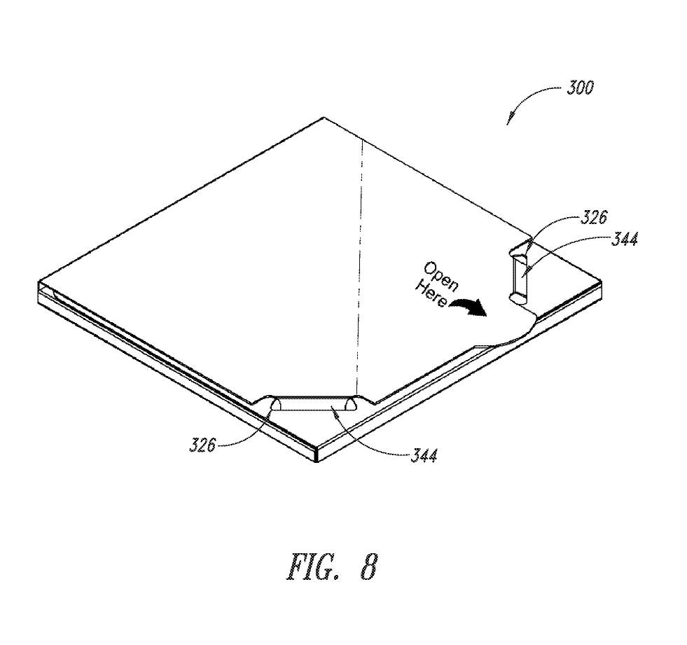

The operator securely closes package by inserting one or more locking tabs 344 into one or more respective locking slots 326. FIG. 8 shows an embodiment of a securely closed suture package.

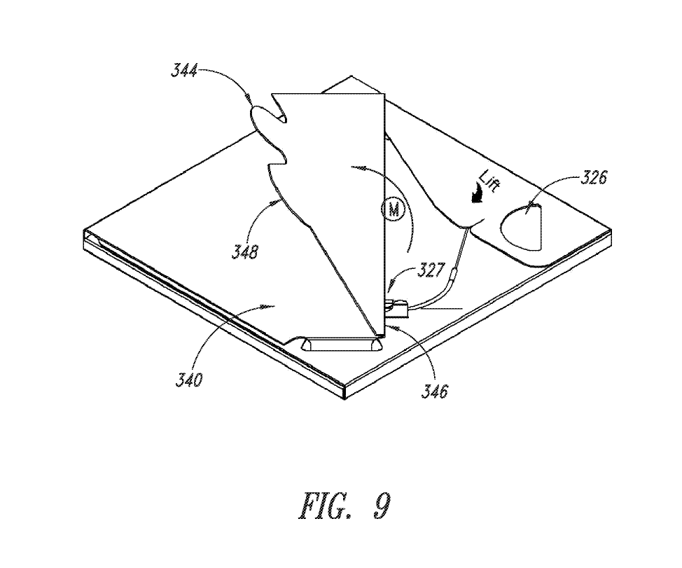

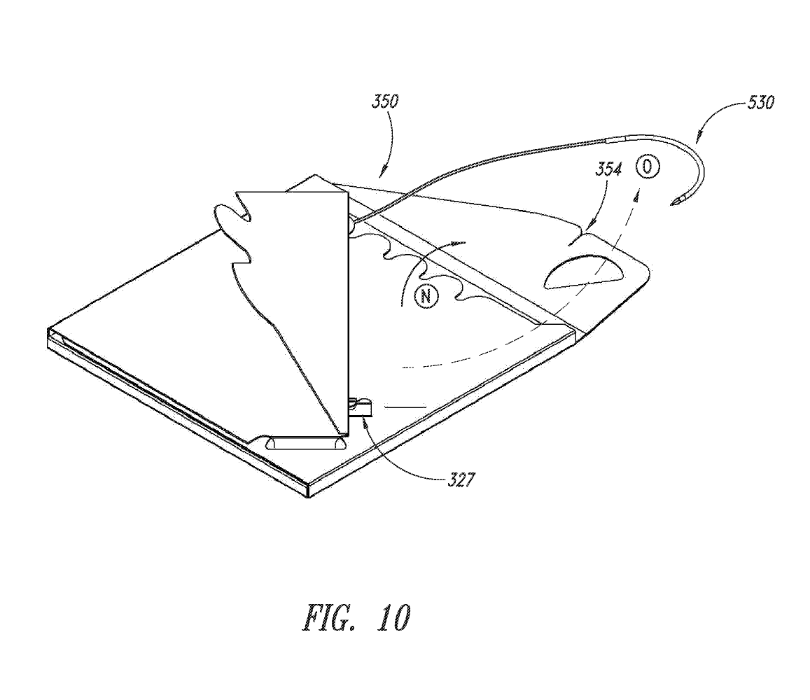

FIGS. 9 and 10 show example steps in a process of opening the package. The package may be opened by folding the left panel 340 along the fold indicator 346 in the direction indicated by the arrow M and lifting a locking tab 344 out from the locking slot 326 while leaving one or more locking tabs 344 engaged with their respective locking slot 326. In some embodiments, the package may be opened by disengaging each locking tab 344 from each respective locking slot 326, thereby unfolding the whole panel 340.

After unfolding the left panel 340 along the fold indicator 346, the operator may unfold the top panel 350 as indicated by arrow N and then remove the needle or needles 530 from the needle nest 327 and the positioning gates 329 as shown by arrow 0. Finally, the operator may pull the entire suture 500 out from the package. Preferably, during the removal process the operator handles the needle 530 and sutures 500 with forceps.

The present disclosure provides the following exemplary embodiments:
1) An apparatus for holding a suture, the apparatus comprising:
   a card including a base and a plurality of panels projecting outwardly from the base, the panels being adapted to fold to form the card into a box-shaped package;
   the base being adapted to mount on a fixture when the card is in an unfolded configuration for receiving the suture;
   the box-shaped package including a plurality of walls enclosing an internal cavity for containing the suture;
   one of the plurality of panels including a slit adapted to retain a portion of the suture proximate a needle while the suture is being placed in the apparatus;
   one of the plurality of panels including a needle nest adapted to capture the needle after the remainder of the suture has been placed into the apparatus; and
   one of the plurality of panels being adapted to facilitate bending whereby a user can open a portion of the box-shaped package to access the needle and remove the needle and suture therefrom.
2) The apparatus of embodiment 1, further comprising a fold indicator extending along an edge of the base between the base and a corresponding one of the plurality of panels.
3) The apparatus of embodiments 1-2, further comprising a locking mechanism adapted to securely close the box-shaped package.
4) The apparatus of embodiment 3, wherein the locking mechanism includes a locking tab on a first of the plurality of panels and a locking slot on a second of the plurality of panels, the locking tab being configured to engage with the locking slot when the card is folded to form the box-shaped package.
5) The apparatus of embodiments 1-4, wherein the needle nest includes opposing fingers for holding one or more needles.
6) The apparatus of embodiment 5, wherein the opposing fingers are interleaved.
7) The apparatus of embodiments 1-6, wherein the needle nest is located adjacent a slot within the panel for facilitating the bending of the panel and the opening of the needle nest.
8) The apparatus of embodiments 1-7, wherein the slit is in a panel different from the panel having the needle nest.
9) The apparatus of embodiment 8, wherein one of the plurality of panels includes a first suture positioning structure.
10) The apparatus of embodiment 9, wherein at least one of the plurality of panels includes a second suture positioning structure.
11) The apparatus of embodiment 10, wherein the second suture positioning structure opposes the first suture positioning structure.
12) The apparatus of embodiment 11, wherein one of the plurality of panels includes a suture holding tab and a cutout.
13) The apparatus of embodiments 1-12, wherein the base includes a plurality of apertures adapted to receive pins of a mounting fixture.
14) A system for packaging a suture, the system comprising:
   a mounting fixture comprising a plurality of pins;
   a suture packaging card having a base comprising a plurality of apertures;
   the pins on the mounting fixture being configured to pass through the apertures in the base of the suture packaging card and to receive a coil of the suture material to be packaged within the suture packaging card; and
   the suture packaging card comprising a plurality of panels projecting outwardly from the base, the panels being adapted to fold over the base to form the card into a box-shaped package for retaining the suture.
15) The system of embodiment 14, wherein the pins on the mounting fixture are substantially straight such that the suture packaging card can slide on and off the mounting fixture.
16) The system of embodiment 15, wherein the pins on the mounting fixture are adapted for receiving a coil of the suture material.
17) The system of embodiment 14, wherein the mounting fixture further comprises a mounting plate being adapted to retain the pins, the pins being adapted to retain the suture packaging card on the mounting fixture and being adapted to receive a coil of the suture material.
18) The system of embodiment 17, further comprising a mounting post, the mounting plate being coupled to the mounting post.
19) The system of embodiment 14, wherein one of the plurality of panels includes a needle nest adapted to capture one or more needles after the remainder of the suture has been placed into the suture packaging card.
20) The system of embodiment 19, wherein one of the plurality of panels is adapted to facilitate bending whereby a user can open a portion of the box-shaped package to access the one or more needles and remove the one or more needle and sutures therefrom.
21) The system of embodiments 14-20, further comprising a fold indicator extending along an edge of the base between the base and a corresponding one of the plurality of panels.
22) The system of embodiments 14-21, further comprising a locking mechanism adapted to securely close the box-shaped package, thereby enclosing a cavity.
23) The system of embodiment 22, wherein the locking mechanism includes a locking tab on a first of the plurality of panels and a locking slot on a second of the plurality of panels, the locking tab being configured to engage with the locking slot when the card is folded to form the box-shaped package.
24) The system of embodiment 19, wherein the needle nest includes opposing fingers for holding one or more needles.
25) The system of embodiment 24, wherein the opposing fingers are interleaved.
26) The system of embodiment 19, wherein the needle nest is located adjacent a slot within the panel for facilitating the bending of the panel and the opening of the needle nest.
27) The system of embodiment 19, further comprising a slit in one of the panels, the slit being adapted to retain a portion of the suture proximate a needle while the suture is being placed in the apparatus and the slit being in a panel different from the panel having the needle nest.
28) The system of embodiments 14-27, wherein one of the plurality of panels includes a first suture positioning structure.
29) The system of embodiment 28, wherein one of the plurality of panels includes a second suture positioning structure.
30) The system of embodiment 29, wherein the second suture positioning structure opposes the first suture positioning structure.
31) The system of embodiments 14-30, wherein one of the plurality of panels includes a suture holding tab and a cutout.
32) The system of embodiments 14-31, wherein the base includes the same number of apertures as the pins on the mounting fixture.
33) A method of packaging a suture, the method comprising the steps of:
   mounting a suture packaging card onto a mounting fixture with a portion of the mounting fixture passing through the suture packaging card;
   wrapping a suture into a coil around the portion of the mounting fixture that was passed through the suture packaging card;
   securing the suture coil to the suture packaging card;
   removing the suture packaging card from the mounting fixture;
   securing a suture needle to the suture packaging card;
   forming the suture packaging card into a box shape; and
   securely closing the suture packaging box.
34) The method of embodiment 33, wherein the step of wrapping a suture into a coil further includes the steps of:
   retaining a portion of the suture proximate a needle end of the suture in a slit; and
   wrapping the suture around at least one pin to form a coil.
35) The method of embodiments 33-34, wherein the step of securing the suture coil on the suture packaging card further includes the steps of:
   lifting the suture packaging card;
   organizing loops of the suture coil; and
   folding a first panel of the suture packaging card over the suture coil.
36) The method of embodiments 33-35, wherein the step of removing the suture packaging card from the mounting fixture further includes the steps of:
   pinching the loops of the suture coil between a first panel and a base of the suture packaging card;
   removing the suture packaging card from the mounting fixture;
   removing a portion of the suture proximate a needle end of the suture from the slit;
   placing a portion of the suture proximate a needle end of the suture in a suture positioning structure.
   folding a second panel over a top of the first panel; and
   folding a third panel over a top of the second panel.
37) The method of embodiments 33-36, wherein the step of securing a suture needle to the suture packaging card further includes the steps of:
   bending a corner of the suture packaging card;
   opening a needle nest of the suture packaging card; and
   inserting the suture needle into the needle nest.
38) The method of embodiments 33-37, wherein the step of forming the suture packaging card into a box shape further includes the step of:
   folding a fourth panel of the suture packaging card over a base of the suture packaging card to create a wall and an internal cavity of the suture packaging card.
39) The method of embodiments 33-38, wherein the step of securely closing the suture packaging box further includes the step of engaging a locking tab of the fourth panel with a locking slot of the second panel.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An apparatus for holding a suture, the apparatus comprising:
a card including a base and a plurality of panels projecting outwardly from the base, the panels being adapted to fold to form the card into a box-shaped package;
the base being adapted to mount on a fixture when the card is in an unfolded configuration for receiving the suture;
the box-shaped package including a plurality of walls enclosing an internal cavity for containing the suture;
one of the plurality of panels including a slit adapted to retain a portion of the suture proximate a needle while the suture is being placed in the apparatus;
one of the plurality of panels including a needle nest adapted to capture the needle after the remainder of the suture has been placed into the apparatus; and
one of the plurality of panels being adapted to facilitate bending whereby a user can open a portion of the box-shaped package to access the needle and remove the needle and suture therefrom.

2. The apparatus of claim 1, further comprising a fold indicator extending along an edge of the base between the base and a corresponding one of the plurality of panels.

3. The apparatus of claim 2, further comprising a locking mechanism adapted to securely close the box-shaped package.

4. The apparatus of claim 1, wherein the needle nest includes opposing fingers for holding one or more needles.

5. The apparatus of claim 1, wherein the slit is in a panel different from the panel having the needle nest.

6. The apparatus of claim 8, wherein one of the plurality of panels includes a first suture positioning structure.

7. The apparatus of claim 1, wherein the base includes a plurality of apertures adapted to receive pins of a mounting fixture.

8. A system for packaging a suture, the system comprising:
a mounting fixture comprising a plurality of pins;
a suture packaging card having a base comprising a plurality of apertures;
the pins on the mounting fixture being configured to pass through the apertures in the base of the suture packaging card and to receive a coil of the suture material to be packaged within the suture packaging card; and
the suture packaging card comprising a plurality of panels projecting outwardly from the base, the panels being adapted to fold over the base to form the card into a box-shaped package for retaining the suture.

9. The system of claim 8, wherein the pins on the mounting fixture are substantially straight such that the suture packaging card can slide on and off the mounting fixture.

10. The system of claim 8, wherein one of the plurality of panels including a needle nest adapted to capture one or more needles after the remainder of the suture has been placed into the suture packaging card.

11. The system of claim 8 further comprising a fold indicator extending along an edge of the base between the base and a corresponding one of the plurality of panels.

12. The system of claim 8, further comprising a locking mechanism adapted to securely close the box-shaped package, thereby enclosing a cavity.

13. The apparatus of claim 8, wherein one of the plurality of panels includes a first suture positioning structure, or one of the plurality of panels includes a suture holding tab and a cutout.

14. The system of claim 8, wherein the base includes the same number of apertures as the pins on the mounting fixture.

15. A method of packaging a suture, the method comprising the steps of:
mounting a suture packaging card onto a mounting fixture with a portion of the mounting fixture passing through the suture packaging card;
wrapping a suture into a coil around the portion of the mounting fixture that was passed through the suture packaging card;
securing the suture coil to the suture packaging card;
removing the suture packaging card from the mounting fixture;
securing a suture needle to the suture packaging card;
forming the suture packaging card into a box shape; and
securing closing the suture packaging box.
